(19) United States
(12) Patent Application Publication
(10) Pub. No.: US 2005/0187158 A1
(43) Pub. Date: Aug. 25, 2005

(54) Pharmaceutical composition

(76) Inventor: Mats G. Ranby, Vrcta Kloster (SE)

Correspondence Address: FISH & RICHARDSON P.C., PO BOX 1022, MINNEAPOLIS, MN, 55440-1022, US

(21) Appl. No.: 11/040,510

(22) Filed: Jan. 21, 2005

Related U.S. Application Data
(60) Provisional application No. 60/538,730, filed on Jan. 22, 2004.

(51) Int. Cl. A61K 38/18

(52) U.S. Cl. 514/12

(57) ABSTRACT

The invention relates to a pharmaceutical composition to be applied superficially, whereby the pharmaceutical composition comprises erythropoietin (EPO) and a pharmaceutically acceptable diluent, adjuvant, or carrier.

### CROSS REFERENCE TO RELATED APPLICATIONS

This application claims priority under 35 U.S.C. §119(e) of U.S. Application No. 60/538,730 filed Jan. 22, 2004.

### TECHNICAL FIELD

The present invention relates to a pharmaceutical composition.

### BACKGROUND

In the hematopoietic system of mammalians, red blood cells are continuously produced. The production of these red blood cells is stimulated by the polypeptide erythropoietin (EPO). Naturally-occurring EPO is an acidic glycoprotein.

It is known to use systemically pharmaceutical compositions comprising glycosylated recombinant human EPO as a therapeutic agent to treat anaemia. Without glycosylation, the EPO is degraded quickly in the liver and is thereby removed from the bloodstream.

The problem to be solved by the present invention is to provide a further pharmaceutical composition comprising EPO, a method for the manufacture of the pharmaceutical composition and a method to treat a human being or an animal.

### SUMMARY

According to the invention, a pharmaceutical composition is provided. The pharmaceutical composition is to be applied superficially (e.g., topically). The pharmaceutical composition comprises erythropoietin (EPO) and a pharmaceutically acceptable diluent, adjuvant, or carrier.

In one aspect, the invention provides a pharmaceutical composition to be applied locally and superficially (e.g,. topically). Such a pharmaceutical composition comprises erythropoietin (EPO) and a pharmaceutically acceptable diluent, adjuvant, or carrier. Generally, the pharmaceutical composition is applied for the treatment of a disease, disorder, or surgical lesion of a human being or an animal.

In another aspect, the invention provides a method to treat a human being or an animal having a disease, disorder, or surgical lesion. Such a method includes administering locally and superficially (e.g., topically) a pharmaceutical composition comprising erythropoietin (EPO) and a pharmaceutically acceptable diluent, adjuvant, or carrier to a diseased, disordered, or surgically injured area.

Typically, the disease or disorder is a disease or disorder of the skin or the eye of a human being or an animal. The disease or disorder can be a conjunctivitis, a wound, a bedsore, a burn, an inflammation, an eczema, or a skin disorder accompanied by necrosis, by a dermatitis, by psoriasis or by diabetes mellitus. A representative disorder of the skin is a lack of growth or colouring of hair.

The EPO in a pharmaceutical composition of the invention can be in a form that is more rapidly cleared from the circulation of a human being or an animal than naturally-occurring EPO. In certain embodiments, the EPO is not glycosylated. In certain other embodiments, the EPO is not PEGylated. In certain additional embodiments, the EPO is not glycosylated and is not PEGylated. In certain embodiments, the EPO is completely formed by amino acid residues that are not modified.

Generally, a pharmaceutical composition of the invention can be an ointment, a cream, a gel, a glycerogelatine, a paste, a plaster, a sprayable composition, a powder, an emulsion, or a lotion. A pharmaceutical composition of the invention generally includes from about 0.01 μg to 100 mg of EPO per 1000 mg of the composition. In certain embodiments, the EPO is comprised in the pharmaceutical composition in a concentration that allows an administration of 50 to less than 500 units of EPO per week (e.g., 50 to less than 200 units of EPO per week).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the detailed description and the claims.

### DETAILED DESCRIPTION OF INVENTION

The present invention is based on the discovery that EPO can be administered superficially (e.g., topically) to provide a local therapeutic effect. The reason for this therapeutic effect is due to an accelerating effect by EPO on differentiation and proliferation processes of a large number of different progenitor cells. It has been found that these progenitor cells have receptors for EPO. EPO stimulates endothelial progenitor cells as well as mesenchymal progenitor cells such as human dermal stem cells and human hair follicle progenitor cells.

The EPO according to the invention may be a protein having a part or all of the primary structure of naturally-occurring EPO. The EPO according to the invention possesses the above-mentioned therapeutic effect. Therefore, the EPO may be identical to naturally-occurring EPO or may be a fragment or derivative thereof, e.g. with one or several point mutations, deletions, insertions, or truncations with respect to the naturally-occurring EPO.

For example, EPO according to the invention can have at least 70% (e.g., 75%, 80%, 85%, 90%, 95%, 99%, or 100%) sequence identity to a human EPO. The amino acid sequence of several human EPO polypeptides can be found, for example, in GenBank Accession Nos. AAC78791, AAF23134, AAF23132, AAF17572, and AAF23133. Sequence identity can be calculated using any of a number of computer programs. For example, BLAST (Altschul et al., 1990, J. Mol. Biol., 215:403-410 and at ncbi.nlm.nih.gov on the World Wide Web), DNA Star (at dnastar.com on the World Wide Web), or the GCG Sequence Analysis Software Package (Wisconsin Package) from the Genetics Computer Group, Inc. can be used to determine the percent sequence identity between two nucleotide or amino acid sequences.

The advantage of the superficial (e.g., topical) application is that the EPO is not applied systemically and, therefore, side effects associated with the systemic application can be avoided. Since the EPO only has to have a local effect, the dosage with respect to the total body mass can be low.

The pharmaceutical composition may be applied for the treatment of a disease, disorder, or surgical lesion of a human being or an animal. The disease or disorder may be a disease or disorder of the skin or of the eye. Surprisingly it was found that the superficial (e.g., topical) application of EPO results in an accelerated healing of such a disease or disorder and of surgical lesions. Preferably the treatment is a local treatment.

In one embodiment, the EPO is in a form that is more rapidly cleared from the circulation of a human being or an animal than naturally-occurring EPO. The advantage of this feature is that it allows superficial application of EPO for local treatment, including treatment at high dosages of EPO, with minimal or no risk of systemic side effects. The treatment may affect only tissue with a common lymph drain, but may allow effective EPO activities to be reached also at some depth below the point or area of superficial application. With this embodiment of the invention, application of low dosages of EPO will further reduce the already low risk of systemic side effects. For those skilled in the art, it follows that any form of EPO that is quickly removed from circulation and/or inactivated, regardless of the biological mechanism, will function in the above described embodiment of the invention.

The form of the EPO that is more rapidly cleared from the circulation of a human being or an animal than naturally-occurring EPO may be an EPO that is not glycosylated, especially not PEGylated. The EPO may even completely be formed by amino acid residues that are not modified. It is not necessary that the EPO comprised in the pharmaceutical composition is a glycoprotein as is the naturally-occurring EPO. Surprisingly, it was found that the non-glycosylated EPO causes an accelerated healing of the diseases, disorders, or surgical lesions. If non-glycosylated EPO is applied systemically, it is disintegrated quickly when passing through the liver.

When applying such a rapidly-cleared form of the EPO superficially, it will predominantly have an effect only locally. As such EPO diffuses away from the point of application it will, e.g. via the lymph, reach the bloodstream and the liver and be disintegrated quickly. The biological activity of the EPO is thereby lost and with that, its ability to cause systemic effects including systemic side effects.

An additional advantage of the fact that no glycosylation or other modification of amino acid residues is necessary is that the EPO may be cost-effectively produced in large amounts by gene technology in bacteria and without the need for ex-vivo chemical modification. The bacteria may be E. coli.

The pharmaceutical composition may be an ointment, a cream, a gel, a glycerogelatine, a paste, a plaster, a sprayable composition, a powder, an emulsion, or a lotion. The pharmaceutical composition may comprise further components that may contribute to a healing. The disease or disorder may be a conjunctivitis, a wound, a bedsore, a burn, an inflammation, an eczema, or a skin disorder accompanied by necrosis, by a dermatitis, by psoriasis, or by diabetes mellitus. The skin disorder accompanied by diabetes mellitus may be, for example, an open leg wound. The disorder of the skin may also be a lack of growth or colouring of hair.

The EPO may be comprised in the pharmaceutical composition in a concentration that allows an administration of 50 to less than 500 units of EPO per week, especially 50 to less than 200 units of EPO per week. This is a much lower dosage than that usually used for a systemic application. For a systemic application, up to 20,000 units are applied per week.

The invention also relates to a use of EPO for the manufacture of a pharmaceutical composition to be applied superficially for the treatment of a disease, disorder, or surgical lesion of a human being or an animal.

The invention also relates to a method to treat a human being or an animal having a disease, disorder, or surgical lesion comprising the superficial local administration of a pharmaceutical composition according to the invention on a diseased, disordered, or surgically-injured area. The disease or disorder may be a disease or disorder of the skin or the eye. EPO may be present in a pharmaceutical composition of the invention in an amount from about 0.01 μg to 100 mg per 1000 mg of the composition. The treatment may be performed by administering the pharmaceutical composition in a dosage of 50 to less than 500 units of EPO per week, especially 50 to less than 200 units of EPO per week.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the invention described in the claims.

### EXAMPLE 1

### A Pharmaceutical Composition

Recombinant human erythropoietin (hEPO) can be obtained as a non-glycosilated, non-soluble or soluble protein from cultured bacteria using standard techniques as described in Molecular Cloning: a laboratory manual, Sambrook et al., 3rd ed., 2001, Cold Spring Harbour Laboratory Press, Cold Spring Harbour, N.Y. A DNA sequence coding for hEPO may be fused with other DNA sequences. These DNA sequences may resemble sequences of protease cleavage sites and sequences of polypeptides that may facilitate the purification of the protein, e.g. affinity tags. The hEPO can be isolated from so-called “inclusion bodies” (IBs) that may be formed during expression in bacteria.

A pharmaceutical composition according to the invention can be provided as follows:

hEPO is expressed in E. coli as a fusion protein comprising the sequence of hEPO (SEQ ID NO:1; 167 amino acids) preceded at its N-terminus by the sequences of an enterokinase cleavage site and at least one affinity tag which may be a Strep-Tag® (IBA GmbH, Göttingen, Germany). One suitable affinity tag that can be used results in an additional 55 amino acids in addition to the EPO protein. During the expression of hEPO, IBs are formed. The expressing bacteria are pelleted and disintegrated. The fusion protein is isolated from IBs by treatment with BugBuster® Protein Extraction Reagent solution (Novagen, Madison, USA) at a concentration of 5 ml BugBuster® Protein Extraction Reagent solution per 1 g wet bacterial cell pellet (=starting volume) for 30 minutes at room temperature according to manufacturer's instructions. The resulting suspension is pelleted by centrifugation at 20,000×g for 20 minutes at 4° C. The pellet is resuspended in 1-fold starting volume of undiluted BugBuster® Protein Extraction Reagent solution.

The resulting slurry is diluted in 6-fold starting volume of 1:10 diluted BugBuster® Protein Extraction Reagent solution. Proteases may be inhibited by the addition of the general protease inhibitor Complete® (Boehringer Mannheim, Mannheim, Germany). Genomic DNA is degraded by the addition of Benzonase (Purity Grade >99%, Merck KGaA, Darmstadt, Germany) as DNA-degrading enzyme. IBs are obtained by a subsequent centrifugation at 5,000×g for 15 minutes at 4° C. The IBs that are contained in the resulting pellet are further purified by at least one washing step by resuspending the pellet using 0.5-fold starting volume of 1:10 diluted BugBuster® Protein Extraction Reagent solution and subsequent centrifugation at 5,000×g for 15 minutes at 4° C.

Purified IBs are obtained by a final washing step comprising the resuspension of the pellet in 0.5-fold starting volume of 1:10 diluted BugBuster® Protein Extraction Reagent solution and subsequent centrifugation at 16,000×g for 15 minutes at 4° C. The purified IBs are solubilized by resuspending them thoroughly at least one time with 0.5-fold starting volume of the following urea- and detergent-containing sodium-phosphate buffer: 1.6 mmol/l NaH₂PO₄, 8.4 mmol/l Na₂HPO₄, 6 mmol/l DTT, 2 mol/l urea, 0.002% Brij® 58 P (Fluka, Sigma-Aldrich Chemie GmbH, Steinheim, Germany), pH 8.0. Afterwards, an insoluble portion of the IBs is spun down at at least 30,000×g for 15 minutes at 4° C. The supernatant contains the solubilized fusion protein.

The fusion protein is separated from contaminating protein fractions by affinity purification using a Strep-tag® column (IBA GmbH, Göttingen, Germany) on which the fusion protein is bound. Binding to the column is performed in the presence of the above sodium-phosphate buffer. Bound fusion protein is eluted using the following elution buffer: one part 1 mol/l Tris-Cl (pH 8.0), 1.5 mol/l NaCl, 10 mmol/l EDTA, 25 mmol/l desthiobiotin and nine parts of the above-described sodium-phosphate buffer (pH 8.0). The fusion protein may be cleaved at the enterokinase cleavage site by digestion with enterokinase, preferentially directly on the column or after the above-mentioned elution. The resulting protein is dialyzed against a physiologically-tolerated buffer, preferably PBS (137 mmol/l NaCl, 2.7 mmol/l KCl, 4.3 mmol/l Na₂HPO₄, 1.47 mmol/l KH₂PO₄, pH 7.4) containing 2 mol/l urea and incorporated into a standard cream formulation containing up to 12% urea. Alternatively, the buffer and/or the cream formulation may lack urea. A standard cream formulation such as “Cremaba Plus HT” (Spinnrad®, Certus Handels GmbH, 22848 Norderstedt, Germany) can be used. “Cremaba Plus HT” has the following ingredients: Aqua, Caprylic/Capric Triglyceride, Pentylene Glycol, Hydrogenated Lecithin, Butyrospermum Parkii, Glycerin, Squalane, Ceramide 3.

Alternative to the above-described expression of EPO as a Strep-Tag® fusion protein, hEPO can also be expressed in E.coli as a glutathione S-transferase fusion protein as described in Bill et al., 1995, Biochem. Biophys. Acta, 1261:35-43. For this purpose, the vector pGEX (Amersham Biosciences, Freiburg, Germany) can be used.

Alternative to the above-described solubilization of purified IBs to obtain a solution that can be incorporated into an ointment, a cream, or a gel, purified IBs can be incorporated directly into an ointment, a cream, or a gel.

Alternative to the above-described IBs, soluble EPO can be produced in bacteria and used in a pharmaceutical composition of the invention. Soluble EPO can be purified from bacteria using routine chromatography methods.

SEQ ID NO:1

MAPPRLICDSRVLERYLLEAKEAENITTGCAEHCSLNENITVPDTKVNFY
AWKRMEVGQQAVEVWQGLALLSEAVLRGQALLVNSSQPWEPLQLHVDKAV
SGLRSLTTLLRALRAQKEAISPPDAASAAPLRTITADTFRKLFRVYSNFL
RGKLKLYTGEACRTGDR

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A pharmaceutical composition to be applied superficially, wherein the pharmaceutical composition comprises erythropoietin (EPO) and a pharmaceutically acceptable diluent, adjuvant, or carrier.

2. The pharmaceutical composition according to claim 1, whereby the pharmaceutical composition is to be applied for the treatment of a disease, disorder, or surgical lesion of a human being or an animal.

3. The pharmaceutical composition according to claim 2, whereby the disease or disorder is a disease or disorder of the skin or the eye of a human being or an animal.

4. The pharmaceutical composition according to claim 2, whereby the treatment is a local treatment.

5. The pharmaceutical composition according to claim 1, whereby the EPO is in a form that is more rapidly cleared from the circulation of a human being or an animal than naturally-occurring EPO.

6. The pharmaceutical composition according to claim 1, whereby the EPO is not glycosylated.

7. The pharmaceutical composition according to claim 1, whereby the EPO is not PEGylated.

8. The pharmaceutical composition according to claim 1, whereby the EPO is not glycosylated and is not PEGylated.

9. The pharmaceutical composition according to claim 1, whereby the EPO is completely formed by amino acid residues that are not modified.

10. The pharmaceutical composition according to claim 1, whereby the pharmaceutical composition is an ointment, a cream, a gel, a glycerogelatine, a paste, a plaster, a sprayable composition, a powder, an emulsion, or a lotion.

11. The pharmaceutical composition according to claim 2, whereby the disease or disorder is a conjunctivitis, a wound, a bedsore, a burn, an inflammation, an eczema, or a skin disorder accompanied by necrosis, by a dermatitis, by psoriasis or by diabetes mellitus.

12. The pharmaceutical composition according to claim 11, whereby the disorder of the skin is a lack of growth or colouring of hair.

13. The pharmaceutical composition according to claim 1, wherein the pharmaceutical composition comprises from about 0.01 μg to 100 mg of EPO per 1000 mg of the composition.

14. The pharmaceutical composition according to claim 1, whereby the EPO is comprised in the pharmaceutical composition in a concentration that allows an administration of 50 to less than 500 units of EPO per week.

15. The pharmaceutical composition according to claim 1, whereby the EPO is comprised in the pharmaceutical composition in a concentration that allows an administration of 50 to less than 200 units of EPO per week.

16. A method to treat a human being or an animal having a disease, disorder, or surgical lesion comprising administering locally and superficially a pharmaceutical composition according to claim 1 to a diseased, disordered, or surgically injured area.

17. The method according to claim 16, whereby the disease or disorder is a disease or disorder of the skin or the eye.

18. The method according to claim 16, whereby the pharmaceutical composition is administered in a dosage of 50 to less than 500 units of EPO per week.

19. The method according to claim 16, whereby the pharmaceutical composition is administered in a dosage of 50 to less than 200 units of EPO per week.

20. The method according to claim 16, whereby the EPO is in a form that is more rapidly cleared from the circulation of a human being or an animal than naturally-occurring EPO.

21. The method according to claim 16, whereby the EPO is not glycosylated.

22. The method according to claim 16, whereby the EPO is not PEGylated.

23. The method according to claim 16, whereby the EPO is not glycosylated and is not PEGylated.
